(19) United States
(12) Patent Application Publication
(10) Pub. No.: US 2024/0207323 A1
(43) Pub. Date: Jun. 27, 2024

(54) METHOD FOR TREATING ACUTE RESPIRATORY DISTRESS SYNDROME (ARDS) USING MESENCHYMAL LINEAGE PRECURSOR OR STEM CELLS

(71) Applicant: MESOBLAST INTERNATIONAL SÁRL, Meyrin (CH)

(72) Inventor: Silviu ITESCU, Melbourne, VIC (AU)

(21) Appl. No.: 18/556,845

(22) PCT Filed: Apr. 22, 2022
(86) PCT No.: PCT/IB2022/053763

§ 371 Date: Oct. 23, 2023

(30) Foreign Application Priority Data
Apr. 23, 2021 (AU) 2021901214
Jul. 15, 2021 (AU) 2021902180
Feb. 9, 2022 (AU) 2022900260
Feb. 18, 2022 (AU) 2022900372

(51) Int. Cl. A61K 35/28; A61K 31/573; A61P 31/14

(52) U.S. Cl. CPC A61K 35/28; A61K 31/573; A61P 31/14

(57) ABSTRACT

The present disclosure relates to methods for treating or preventing Acute Respiratory Distress Syndrome (ARDS) in a subject in need thereof, the method comprising administering to the subject a composition comprising mesenchymal lineage precursor or stem cells (MLPSCs).

### FIELD OF THE DISCLOSURE

The present disclosure relates to methods for treating or preventing Acute Respiratory Distress Syndrome (ARDS) in a subject in need thereof.

### BACKGROUND

Respiratory ailments, associated with a variety of conditions such as viral infection are problematic in the general population. In many cases they are accompanied by inflammation, which aggravates the condition of the lungs and can result in Acute Respiratory Distress Syndrome (ARDS).

There remains an unmet therapeutic need in patients with Acute Respiratory Distress Syndrome (ARDS), particularly when secondary to viral infection with new treatment options being required.

### SUMMARY OF THE DISCLOSURE

The present inventors identified that corticosteroid treatment with dexamethasone in ARDS does not provide protection against death, in particular in patients that are less than 65 years of age. The present inventors surprisingly identified that this deficiency is remedied by administering MLPSCs with dexamethasone. These findings suggest that treatment of ARDS can be improved, in particular in relation to prospects for survival, by treating ARDS patients with a corticosteroid and MLPSCs. Accordingly, in a first example, the present disclosure relates to a method of treating or preventing Acute Respiratory Distress Syndrome (ARDS) in a human subject in need thereof, the method comprising administering to the subject a corticosteroid and a composition comprising mesenchymal lineage precursor or stem cells (MLPSCs). In an example, the subject is less than 65 years old.

The present inventors have also identified an effective method of treating subjects with Acute Respiratory Distress Syndrome (ARDS) that are less than 65 years of age by administering mesenchymal lineage precursor or stem cells (MLPSCs) to these subjects. Accordingly, in another example, the present disclosure relates to a method of treating or preventing Acute Respiratory Distress Syndrome (ARDS) in a human subject in need thereof, the method comprising administering to the subject a composition comprising mesenchymal lineage precursor or stem cells (MLPSCs), wherein the subject is less than 65 years old. In an example, the method further comprises administering a corticosteroid.

The findings of the present inventors suggest that subjects less than 65 years old can be selected for effective treatment according to a method disclosed herein. Accordingly, in an example, the present disclosure relates to a method of treating or preventing Acute Respiratory Distress Syndrome (ARDS) in a human subject in need thereof, the method comprising selecting a subject with ARDS who is less than 65 years old and, administering to the subject a composition comprising mesenchymal lineage precursor or stem cells (MLPSCs). In an example, subjects are selected that are less than 65 years of age and taking a corticosteroid.

In the above examples, the subject may be less than 60 years old. In another example, the subject may be between 18 and 65 years old. In another example, the subject may be between 18 and 60 years old.

In an example, the subjects ARDS is moderate or severe.

In an example, the subject is taking a corticosteroid prior to administering a cellular composition disclosed herein. In an example, the corticosteroid is dexamethasone.

In an example, the subject is on a ventilator. For example, the subject can be mechanically ventilated prior to administering MLPSCs. In an example, the subject is taken off the ventilator after treatment. In an example, the subject is taken off a ventilator within 60 days of treatment.

In an example, the ARDS is caused by a viral infection. The viral infection may be caused, for example, by a rhinovirus, influenza virus, respiratory syncytial virus (RSV) or a coronavirus.

In one example, the ARDS is caused by a coronavirus infection. The coronavirus may be, for example, Severe Acute Respiratory Syndrome coronavirus (SARS-CoV), Middle East Respiratory Syndrome coronavirus (MERS-CoV), COVID-19, 229E, NL63, OC43, or KHU1. In one example, the coronavirus is SARS-COV, MERS-COV or COVID-19 (SARS-COV-2).

In an example, the ARDS is caused by a thrombosis such as a venous thrombosis or an arterial thrombosis. In another example, the ARDS is caused by a pulmonary embolism.

In an example, a treated subjects risk of mortality is reduced after treatment. In an example, a treated subjects risk of mortality is reduced between 30 and 60%. In an example, a treated subject has improved 60 day survival.

In an example, improved survival is increased days alive of ventilation. Accordingly, in an example, the reduced risk of mortality is determined in subjects that are breathing unassisted by mechanical ventilation.

In an example, treatment according to the disclosure increases the number of days alive off ventilation. In an example, the increase is observed at day 60 following treatment.

In an example, treatment improves respiratory function. In an example, improved respiratory function is defined as resolution and/or improvement of ARDS as defined by the Berlin criteria at one or more or all of days 7, 14, 21, and 30 following treatment. In an example, the improvement is observed at day 7. In an example, the improvement is observed at day 14. For example, treatment may improve respiratory function as defined by Berlin criteria at day 14 and/or day 21. In an example, improved respiratory function is maintained beyond day 7 relative to baseline respiratory function. In an example, improved respiratory function is maintained at day 14 relative to baseline respiratory function.

In an example, treatment improves clinical outcome. In an example, improvement in clinical outcome is assessed based on a 7-point ordinal scale at baseline and one or more or all of days 7, 14, 21, and 30 and discharge from hospital.

In another example, treatment decreases the level of at least one inflammatory biomarker(s) relative to baseline, wherein the at least one inflammatory biomarker(s) indicate:
reduced neutrophil and macrophage influx into lungs;
reduced inflammasome;
reduced macrophage activation and neutrophil migration to lungs;
reduced T cell influx and activation; or
reduced circulating biomarkers of macrophage and neutrophil inflammation.

In an example, the inflammatory biomarker(s) is one or more of the following:
a CXCR3-binding chemokine, preferably CXCL10, and/or CXCL9;
CCR2-binding chemokine, preferably CCL2, CCL3, and/or CCL7;
IL-6;
IL-8;
TNF;
IL-18;
CCL19;
IL-4;
IL-13;
GM-CSF;
CRP; or
Ferritin.

In an example, treatment reduces CRP and/or ferritin levels within 3 to 14 days of administering MLPSCs.

In an example, the MLPSCs have been cryopreserved and thawed.

In an example, the MLPSCs are culture expanded from an intermediate cryopreserved MLPSCs population. In another example, the MLPSCs are culture expanded for at least about 5 passages. In an example, the MLPSCs express at least 13 pg TNF-R1 per million MLPSCs. In an example, the MLPSCs express about 13 pg to about 44 pg TNF-R1 per million MLPSCs. In an example, culture expanded MLPSCs are culture expanded for at least 20 population doublings. In another example, culture expanded MLPSCs are culture expanded for at least 30 population doublings. In an example, the MLPSCs are mesenchymal stem cells (MSCs). In another example, the MLPSCs are allogeneic. For example, the MLPSCs may be allogeneic MSCs.

In another example, the MLPSCs are modified to carry or express an anti-viral drug or a thrombolytic agent. In an example, the anti-viral drug is Remdesivir. In an example, the thrombolytic agent is selected from the group consisting of Eminase (anistreplase) Retavase (reteplase) Streptase (streptokinase, kabikinase).

In another example, the MLPSCs are genetically modified to express an anti-viral peptide or a nucleic acid encoding the same.

In an example, the composition is administered intravenously.

In an example, the methods of the disclosure encompass administering between 1×10⁷ and 2×10⁸ cells. For example, multiple doses of between 1×10⁷ and 2×10⁸ cells may be administered on days 0, 30, 60 and 90. In an example, the methods of the disclosure encompass administering about 1×10⁸ cells per dose. In an example, the subject is administered two doses.

In an example, the subject receives a second dose within 7 days of being administered a first dose. In an example, the second dose is administered 4 days after the first dose. In an example, a dose comprises 2×10⁶ cells/kg of body weight.

In another example, the composition further comprises Plasma-Lyte A, dimethyl sulfoxide (DMSO), human serum albumin (HSA). In an example, the composition further comprises Plasma-Lyte A (70%), DMSO (10%), HSA (25%) solution, the HSA solution comprising 5% HSA and 15% buffer.

In an example, the composition comprises greater than 6.68×10⁶ viable cells/mL.

### BRIEF DESCRIPTION OF ACCOMPANYING FIGURES

FIG. 1: Cell therapy provides some degree of protection against death at day 60 in all patients. A: All Intention to treat (ITT) patients; B: All per protocol (PP) patients.

FIG. 2: Cell therapy provides protection against death at day 60 in patients <65 years old. A: ITT patients <65 years old; B: ITT patients ≥65 years old.

FIG. 3: Cell therapy provides protection against death at day 60 in patients <65 years old. A: PP patients <65 years old (n=123); B: PP patients ≥65 years old (n=94).

FIG. 4: Cell therapy protects against all cause deaths at day 60 in ITT and PP patients <60 years old who received dexamethasone at baseline. A: ITT patients; B: PP patients.

FIG. 5: Dexamethasone at baseline does not provide protection against death at day 60 in control patients. A: ITT patients <65 years old; B: ITT patients >65 years old.

FIG. 6: Dexamethasone at baseline provides synergistic protection against death at day 60 when combined with cell therapy in patients <65 years old. Furthermore, cell therapy plus dexamethasone outperforms all other treatment arms in mortality reduction through 60 days. A: ITT patients; B: PP patients; C: All treated patients <65 years old on Dexamethasone (n=73).

FIG. 7: Cell therapy plus Dexamethasone: Analysis of Respiratory Function and Clinical Improvement in Exploratory Population <65 years old. Respiratory Function Improvement measured as resolution and/or improvement of ARDS as defined by the Berlin criteria at Days 7, 14, 21, and 30 post-randomizations; Clinical Improvement was assessed based on a 7-point ordinal scale at baseline and on Days 7, 14, 21, and 30 and discharge from hospital. A: Respiratory function improvement in ITT patients <65 years old on Dexamethasone (n=73). B: Clinical improvement in ITT patients <65 years old on Dexamethasone (n=73).

FIG. 8: Analysis of respiratory function improvement in A: patients <65 years old and B: patients ≥65 years old. Respiratory function measured as resolution and/or improvement of ARDS as defined by the Berlin criteria at Days 7, 14, 21, and 30 post-randomizations.

FIG. 9: Cell therapy reduces mortality by 48% through 90 Days in Pre-Specified Analysis <65 years old (n=123).

FIG. 10: Cell therapy increases ventilator-free days alive through 60 Days in Patients <65 years old. A) All treated patients <65 years old (n=123). B) All Treated Patients <65 years old on Dexamethasone (n=73).

FIG. 11: A: CRP levels at baseline and day 3, 7 and 14; B: Ferritin levels at baseline and day 3, 7 and 14; C: D-dimers at baseline and day 3, 7 and 14.

FIG. 12: Patients aged >65 have a greater level of baseline inflammation. Data is fold change in levels.

FIG. 13: Inflammatory biomarker stratified analysis by age group. Data is fold change in levels from baseline.

### DETAILED DESCRIPTION

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

Any example disclosed herein shall be taken to apply mutatis mutandis to any other example unless specifically stated otherwise.

Unless specifically defined otherwise, all technical and scientific terms used herein shall be taken to have the same meaning as commonly understood by one of ordinary skill in the art (e.g., in cell culture, molecular genetics, stem cell therapy, immunology, immunohistochemistry, protein chemistry, and biochemistry).

Unless otherwise indicated, the surgical techniques utilized in the present disclosure are standard procedures, well known to those skilled in the art.

Methods of obtaining and enriching a population of mesenchymal lineage stem or precursor cells are known in the art. For example, enriched populations of mesenchymal lineage stem or precursor cells can be obtained by the use of flow cytometry and cell sorting procedures based on the use of cell surface markers that are expressed on mesenchymal lineage stem or precursor cells.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

### Selected Definitions

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, more preferably +/−5%, of the designated value.

The terms “level” and “amount” are used to define the amount of a particular substance in a cell preparation. For example, a particular concentration, weight, percentage (e.g. v/v %) or ratio can be used to define the level of a particular substance. In an example, the level is expressed in terms of how much of a particular marker is expressed by cells of the disclosure under culture conditions. In an example, expression represents cell surface expression. In another example, the level is expressed in terms of how much of a particular marker is released from cells described herein under culture conditions.

In an example, the level is expressed in pg/ml. In another example, the level is expressed in pg per 10⁶ cells. The level of pg/ml can be converted to pg per 10⁶ cells if required. For example, in the context of TNF-R1, in an example, 200 pg/ml TNF-R1 corresponds to about 23.5 pg of TNF-R1 per 10⁶ cells. In an example, in the context of TNF-R1, in an example, 225 pg/ml TNF-R1 corresponds to about 26.5 pg of TNF-R1 per 10⁶ cells. In an example, 230 pg/ml TNF-R1 corresponds to about 27 pg of TNF-R1 per 10⁶ cells. In another example, 260 pg/ml TNF-R1 corresponds to about 30 pg of TNF-R1 per 10⁶ cells. In another example, 270 pg/ml TNF-R1 corresponds to about 32 pg TNF-R1 per 10⁶ cells and so on.

In an example, the level of a particular marker is determined under culture conditions. The term “culture conditions” is used to refer to cells growing in culture. In an example, culture conditions refers to an actively dividing population of cells. Such cells may, in an example, be in exponential growth phase. For example, the level of a particular marker can be determined by taking a sample of cell culture media and measuring the level of marker in the sample. In another example, the level of a particular marker can be determined by taking a sample of cells and measuring the level of the marker in the cell lysate. Those of skill in the art will appreciate that secreted markers can be measured by sampling the culture media while markers expressed on the surface of the cell may be measured by assessing a sample of cell lysate. In an example, the sample is taken when the cells are in exponential growth phase. In an example, the sample is taken after at least two days in culture.

Culture expanding cells from a cryopreserved intermediate means thawing cells subject to cryogenic freezing and in vitro culturing under conditions suitable for growth of the cells.

In an example, the “level” or “amount” of a particular marker such as TNF-R1 is determined after cells have been cryopreserved and then seeded back into culture. For example, the level is determined after a first cryopreservation of cells. In another example, the level is determined after a second cryopreservation of cells. For example, cells may be culture expanded from a cryopreserved intermediate, cryopreserved a second time before being re-seeded in culture so that the level of a particular marker can be determined under culture conditions.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the singular form “a”, “an” and “the” include singular and plural references unless the context indicates otherwise.

By “isolated” or “purified” it is meant a cell which has been separated from at least some components of its natural environment. This term includes gross physical separation of the cells from its natural environment (e.g. removal from a donor). The term “isolated” includes alteration of the cell's relationship with the neighboring cells with which it is in direct by, for example, dissociation. The term “isolated” does not refer to a cell which is in a tissue section. When used to refer to the population of cells, the term “isolated” includes populations of cells which result from proliferation of the isolated cells of the disclosure.

The terms “passage”, “passaging” or “sub-culture” are used in the context of the present disclosure to refer to known cell culture techniques that are used to keep cells alive and growing under cultured conditions for extended periods of time so that cell numbers can continually increase. The degree of sub-culturing a cell line has undergone is often expressed as “passage number,” which is generally used to refer to the number of times cells have been sub-cultured. In an example, one passage comprises removing non-adherent cells and leaving adherent mesenchymal lineage precursor or stem cells. Such mesenchymal lineage precursor or stem cells can then be dissociated from the substrate or flask (e.g., by using a protease such as trypsin or collagenase), media can be added, optional washing (e.g., by centrifugation) may be performed, and then the mesenchymal lineage precursor or stem cells can be re-plated or reseeded to one or more culture vessels containing a greater surface area in total. The mesenchymal lineage precursor or stem cells can then continue to expand in culture. In another example, methods of removing non-adherent cells include steps of non-enzymatic treatment (e.g., with EDTA). In an example, mesenchymal lineage precursor or stem cells are passaged at or near confluence (e.g., about 75% to about 95% confluence). In an example, the mesenchymal lineage precursor or stem cells are seeded at a concentration of about 10%, about 15%, or about 20% cells/ml of culture medium.

The term “medium” or “media” as used in the context of the present disclosure, includes the components of the environment surrounding cells in culture. It is envisaged that the media contributes to and/or provides the conditions suitable to allow cells to grow. Media may be solid, liquid, gaseous or a mixture of phases and materials. Media can include liquid growth media as well as liquid media that do not sustain cell growth. Exemplary gaseous media include the gaseous phase that cells growing on a petri dish or other solid or semisolid support are exposed to.

As used herein, the terms “treating”, “treat” or “treatment” include administering a population of mesenchymal lineage stem or precursor cells and/or progeny thereof and/or soluble factors derived therefrom to thereby reduce or eliminate at least one symptom of ARDS. In an example, treatment includes administering a population of culture expanded mesenchymal lineage stem or precursor cells. In an example, treatment response is determined relative to baseline. In an example, treatment response is determined relative to a control patient population. In an example, treatment improves the subjects ARDS from severe to moderate.

In an example, methods of the present disclosure inhibit disease progression or disease complication in a subject. “Inhibition” of disease progression or disease complication in a subject means preventing or reducing the disease progression and/or disease complication in the subject. Accordingly, in an example, methods of the disclosure inhibit progression of ARDS severity.

The term “prevent” or “preventing” as used herein include administering a population of mesenchymal lineage stem or precursor cells and/or progeny thereof and/or soluble factors derived therefrom to thereby stop or inhibit the development of at least one symptom of ARDS.

The term “subject” as used herein refers to a human subject. For example, the subject can be an adult. Terms such as “subject”, “patient” or “individual” are terms that can, in context, be used interchangeably in the present disclosure.

The term “thrombosis” is used herein to refer to the formation of a thrombus or blood clot. In an example, the thrombosis is “arterial thrombosis” where the blood clot develops in an artery. Such blood clots are particularly dangerous to a subject as they can obstruct blood flow to major organs such as the heart or brain. In an example, the thrombosis is “venous thrombosis” where the blood clot develops in a vein.

The term “pulmonary embolism” is used herein to refer to a blockage of an artery in the lungs by a substance that has moved from elsewhere in the body through the bloodstream.

As used herein, the term “genetically unmodified” refers to cells that have not been modified by transfection with a nucleic acid. For the avoidance of doubt, in the context of the present disclosure a mesenchymal lineage precursor or stem cell transfected with a nucleic acid encoding Ang1 would be considered genetically modified.

“C-reactive protein” or “CRP” is an inflammatory mediator whose levels are raised under conditions of acute inflammatory recurrence and rapidly normalize once the inflammation subsides. Circulating CRP levels can be measured in a blood plasma sample to provide a measure of inflammation in a subject.

The term “total dose” is used in the context of the present disclosure to refer to the total number of cells received by the subject treated according to the present disclosure. In an example, the total dose consists of one administration of cells. In another example, the total dose consists of two administrations of cells. In another example, the total dose consists of three administrations of cells. In another example, the total dose consists of four or more administrations of cells. For example, the total dose can consist of two to four administrations of cells.

The term “clinically proven” (used independently or to modify the term “effective”) shall mean that efficacy has been proven by a clinical trial wherein the clinical trial has met the approval standards of U.S. Food and Drug Administration, EMEA or a corresponding national regulatory agency. For example, the clinical study may be an adequately sized, randomized, double-blinded study used to clinically prove the effects of the composition. In an example, a clinically proven effective amount is an amount shown by a clinical trial to meet a specified endpoint. In an example, the end point is protection against death.

Accordingly, the terms “clinically proven efficacy” and “clinically proven effective” can be used in the context of the present disclosure to refer to a dose, dosage regimen, treatment or method disclosed herein. Efficacy can be measured based on change in the course of the disease in response to administering a composition disclosed herein. For example, composition of the disclosure is administered to a subject in an amount and for a time sufficient to induce an improvement, preferably a sustained improvement, in at least one indicator that reflects the severity of ARDS. Various indicators that reflect the severity of ARDS can be assessed for determining whether the amount and time of the treatment is sufficient. Such indicators include, for example, clinically recognized indicators of disease severity or symptoms. In an example, the degree of improvement is determined by a physician, who can make this determination based on signs, symptoms, or other test results. In an example, a clinically proven effective amount improves patient survival. In another example, a clinically proven effective amount reduces a subjects risk of mortality. In another example, a clinically proven effective amount reduces a subjects circulating CRP levels.

### Acute Respiratory Distress Syndrome (ARDS)

The methods of the present disclosure relate to the treatment of acute respiratory distress syndrome (ARDS) by administering a composition disclosed herein. In an example, the method comprises administering a composition comprising MLPSCs. Accordingly, in an example, the composition can comprise MSCs. In an example, the methods of the present disclosure comprise administering a cellular composition disclosed herein, such as a composition comprising MLPSCs, and, a corticosteroid. In this example, the corticosteroid can be administered simultaneously or sequentially with the cellular composition. In an example, the subject has been previously taking a corticosteroid prior to administering a cellular composition disclosed herein. In this example, the corticosteroid can continue to be administered along with the cellular composition.

In an example, the corticosteroid is a long acting or intermediate acting (half-life <36 hours) corticosteroid. In an example, the corticosteroid is long acting (half-life of 36 to 72 hours). In an example, the corticosteroid is dexamethasone. Other examples of corticosteroids include prednisone and methylprednisolone.

The term “acute respiratory distress syndrome (ARDS)” is a type of respiratory failure characterized by widespread inflammation in the lungs, poor oxygenation and non-compliant or “stiff” lungs. The disorder is generally associated with capillary endothelial injury and diffuse alveolar damage.

In an example, the methods of the present disclosure prevent or treat subjects with mild ARDS. In another example, the methods of the present disclosure prevent or treat subjects with moderate ARDS. In another example, the methods of the present disclosure prevent or treat subjects with severe ARDS. In another example, the methods of the present disclosure prevent or treat subjects with moderate or severe ARDS. In an example, the methods of the present disclosure treat subjects with ARDS that require ventilation. For example, the subject can be on a mechanical ventilator.

In an example, severity of ARDS is diagnosed depending on the PaO2/FiO2 ratio. For example, severity of ARDS can be diagnosed as follows: (Mild: 26.6 kPa <PaO2/FiO2≤39.9 kPa; Moderate: 13.3 kPa <PaO2/FiO2≤ 26.6 kPa; Severe: PaO2/FiO2≤13.3 kPa). In an example, severity of ARDS can be diagnosed according to the Berlin definition as summarised in the Table below:

| | |
|---|---|
| Timing | Within 1 week of a known clinical insult or new/worsening |
| | respiratory symptoms. |
| Chest | Bilateral opacities not fully explained by effusions. Lobar/ |
| imaging | lung collapse or nodules. |
| Origin of | Respiratory failure not fully explained by cardiac failure or |
| oedema | fluid overload. |
| | Needs objective assessment (e.g. echocardiography) to |
| | exclude hydrostatic oedema if no risk factor present. |
| Oxy- | Mild-26.6 kPa < PaO2/FiO2 ≤ 39.9 kPa with PEEP or |
| genation | CPAP ≥ 5 cm H₂0 |
| | Moderate-13.3 kPa < PaO2/FiO2 ≤ 26.6 kPa with PEEP or |
| | CPAP ≥ 5 cm H₂0. |
| | Severe- PaO2/FiO2 ≤ 13.3 kPa with PEEP ≥ 5 cm H₂O. |

In another example, severity of ARDS can be diagnosed as follows: mild (PaO2/FiO2 200 to 300 mmHg); moderate (PaO2/FiO2 100 to 200 mmHg); severe (PaO2/FiO2 less than 100 mmHg).

In an example, subjects with moderate to severe ARDS have a circulating CRP level >4 mg/L.

In an example, subjects treated according to the methods of the present disclosure are less than 65 years of age. In another example, the subject is less than 60 years of age. In an example, the subject is at least 18 years old. In an example, the subject is between 18 and 65 years of age. In another example, the subject is between 18 and 60 years of age.

In another example, subjects treated according to the methods of the present disclosure are taking a corticosteroid. For example, the subjects can be taking dexamethasone. In an example, the subject is less than 65 years of age and taking a corticosteroid such as dexamethasone. In an example, the subject is less than 60 years of age and taking a corticosteroid such as dexamethasone. In an example, the corticosteroid is a long acting or intermediate acting (half-life <36 hours) corticosteroid. In an example, the corticosteroid is long acting (half-life of 36 to 72 hours). In an example, the corticosteroid is dexamethasone. Other examples of corticosteroids include prednisone and methylprednisolone.

In another example, the methods of the present disclosure comprise administering a cellular composition disclosed herein and a corticosteroid. In this example, the corticosteroid can be administered simultaneously or sequentially with the cellular composition. In an example, the subject has been previously taking a corticosteroid prior to administering a cellular composition disclosed herein. In this example, the corticosteroid can continue to be administered along with the cellular composition.

In an example, the methods of the present disclosure comprises selecting a subject that is less than 65 years of age for treatment. In another example, the methods of the present disclosure comprise selecting a subject that is less than 65 years of age and taking a corticosteroid for treatment. In another example, the methods of the present disclosure comprise selecting a subject that is less than 65 years of age and taking dexamethasone for treatment. In an example, subjects are selected based on an appropriate questionnaire or line of questioning from a treating clinician asking, for example, for the subjects age and current medications.

Subjects treated according to the present disclosure may have symptoms indicative of ARDS. Exemplary symptoms may include fatigue, trouble breathing, shortness of breath, inability or decreased ability to exercise, coughing with or without blood or mucus, pain when breathing in or out, wheezing, chest tightness, unexplained weight loss, musculoskeletal pain, rapid breathing (tachypnea), and, bluish skin coloration (cyanosis).

In another example, the subject has pneumonia.

In another example, the subject has ARDS secondary to viral infection. In an example, the subjects ARDS is secondary to infection with a rhinovirus, an influenza virus, a respiratory syncytial virus (RSV) or a coronavirus. In an example, the subjects ARDS is secondary to infection with a coronavirus. For example, the subjects ARDS can be secondary to infection with SARS-COV, MERS-COV or COVID-19.

In an example, the subject has one or more of myocarditis, pericarditis, or valvulitis. In an example, the subject has viral induced myocarditis, pericarditis, or valvulitis. For example, the subject can have viral myocarditis.

In an example, ARDS is caused by a viral infection. For example, the ARDS can be caused by a rhinovirus, an influenza virus, a respiratory syncytial virus (RSV) or a coronavirus. In an example, the ARDS can be caused by a coronavirus. For example, the coronavirus can be coronavirus (SARS-CoV), Middle East Respiratory Syndrome coronavirus (MERS-CoV) or COVID-19. In an example, the ARDS is caused by Epstein-Barr virus (EBV) or herpes simplex virus (HSV).

In another example, the ARDS is caused by a thrombosis. In another example, the ARDS is caused by an embolism. In an example, ARDS is caused by a pulmonary embolism.

In another example, the ARDS is secondary to hemophagocytic lymphohistiocytosis (HLH). HLH is a life-threatening disease characterized by lymphocyte and macrophage hyperinflammation. HLH can be triggered by viral infections such as EBV, CMV, HHV. Accordingly, in an example, the HLH is secondary or acquired HLH. For example, the HLH can be secondary to viral infection and lead to the development of ARDS in a subject.

In an example, treatment protects against death or imparts improved survival. In an example, protection against death is determined 60 days after treatment. In another example, protection against death is determined 50 to 70 days after treatment. For example, a treated subjects risk of mortality can be reduced after treatment. In an example, a treated subjects risk of mortality is reduced between 30 and 60%. In an example, a treated subjects risk of mortality is reduced between 40 and 50%. In an example, a treated subjects risk of mortality is reduced by at least 30%. In an example, a treated subjects risk of mortality is reduced by at least 40%.

In an example, treatment according to the methods of the present disclosure reduce a subjects risk of thrombosis. In an example, the subjects risk is reduced relative to a subject that does not receive treatment. In an example, treatment reduces the risk of the thrombosis is arterial thrombosis. Accordingly, in an example, treatment reduces the risk of heart attack or stroke in a subject with ARDS.

In an example, treatment reduces the subjects CRP level. In an example, treatment reduces CRP by at least 100 mg/dl compared to baseline. In another example, treatment reduces CRP by at least 150 mg/dl compared to baseline.

In an example, treatment reduces a subjects circulating CRP levels to 80 mg/dl or lower. In an example, treatment reduces a subjects circulating CRP levels to 60 mg/dl or lower. In another example, treatment reduces a subjects circulating CRP levels to 50 mg/dl or lower. In another example, treatment reduces a subjects circulating CRP levels to CRP to 30 mg/dl or lower. In another example, treatment reduces a subjects circulating CRP levels to CRP to 40 mg/dl or lower. In another example, treatment reduces a subjects circulating CRP levels to 20 mg/dl or lower. In another example, treatment reduces a subjects circulating CRP levels to 10 mg/dl or lower. In another example, treatment reduces a subjects circulating CRP levels to 5 mg/dl or lower. In another example, treatment reduces a subjects circulating CRP levels to 3 mg/dl or lower.

In another example, treatment reduces CRP to between 0.5 mg/dl and 30 mg/dl. In another example, treatment reduces CRP to between 0.5 mg/dl and 10 mg/dl. In an example, treatment reduces CRP and/or ferritin levels within 3 to 14 days of administering MLPSCs.

In another example, the present disclosure encompasses selecting a subject with ARDS for treatment. In an example, the subject has moderate or severe ARDS. In an example, the method comprises selecting a subject with ARDS that is less than 65 years of age. In an example, the method comprises selecting a subject that is less than 65 years of age and taking a corticosteroid. In an example, selected subjects are treated according to a method disclosed herein.

### Inflammatory Biomarkers

In an example, treatment reduces inflammatory biomarker(s) level(s) in the subject. In one example, the decrease of an inflammatory biomarker is indicative of reduced neutrophil and macrophage influx into lungs, for example, a CCR2−binding chemokine such as CCL2, CCL3 and CCL7. In an example, the subject has an decreased level of CCL2. In another example, the subject has an decreased level of CCL3. In another example, the subject has an decreased level of CCL7. In another example, inflammatory biomarker is a CXCR3-binding chemokine such as CXCL10 and CXCL9. In an example, the subject has an decreased level of CXCL10. In another example, the subject has an decreased level of CXCL9.

In another example, the decrease of an inflammatory biomarker is indicative of reduced inflammasome. Inflammasomes are stimulus-induced cytoplasmic multimeric protein complexes. In another example, the decrease of an inflammatory biomarker is indicative of reduced macrophage activation and neutrophil homing to lungs. Examples of inflammatory biomarkers that are indicative of reduced inflammasome and reduced macrophage activation/neutrophil homing to lungs when decreased are IL-6, IL-8, tumour necrosis factor (TNF) and interleukin-18 (IL-18). In an example, the subject has a decreased level of IL-6. In another example, the subject has an decreased level of IL-8. In another example, the subject has an decreased level of TNF. In another example, the subject has an decreased level of IL-18.

In another example, the decrease of an inflammatory biomarker is indicative of reduced T cell influx and activation. Examples of inflammatory biomarkers indicative of reduced T cell influx and activation when decreased are C—C motif chemokine ligand 19 (CCL19), interleukin-4 (IL-4) interleukin-13 (IL-13), and granulocyte-macrophage colony-stimulating factor (GM-CSF). In an example, the subject has an decreased level of CCL19. In another example, the subject has an decreased level of IL-4. In another example, the subject has an decreased level of IL-13. In another example, the subject has an decreased level of GM-CSF.

In another example, the decrease of an inflammatory biomarker is indicative of reduced circulating biomarkers of macrophage and neutrophil inflammation, for example, CRP, ferritin, or D-dimer. In an example, the reduced circulating biomarker is “C-reactive protein” or “CRP”. CRP is an inflammatory mediator whose levels are raised under conditions of acute inflammatory recurrence and rapidly normalize once the inflammation subsides. Circulating CRP levels can be measured in a blood plasma sample to provide a measure of inflammation in a subject.

In an example, treatment reduces the subject's CRP level. In an example, treatment reduces CRP by at least 100 mg/dl compared to baseline. In another example, treatment reduces CRP by at least 150 mg/dl compared to baseline. In an example, treatment reduces a subject's CRP levels by about 0.1 fold. In another example, treatment reduces a subject's CRP levels by about 0.2 fold. In another example, treatment reduces a subject's CRP levels by about 0.3 fold. In another example, treatment reduces a subject's CRP levels by about 0.4 fold. In another example, treatment reduces a subject's CRP levels by about 0.5 fold. In another example, treatment reduces a subject's CRP levels by 0.6 fold. In these examples, treatment reduces a subject's CRP levels by fold change relative to the subject's baseline CRP level.

In another example, the reduced circulating biomarker is ferritin. Ferritin is a blood protein that contains iron. Ferritin levels can be measured in a blood sample to provide a measure of a subject's iron levels. In an example, treatment reduces a subject's ferritin levels. In an example, treatment reduces a subject's ferritin levels by about 0.1 fold. In another example, treatment reduces a subject's ferritin levels by about 0.2 fold. In another example, treatment reduces a subject's ferritin levels by about 0.3 fold. In another example, treatment reduces a subject's ferritin levels by about 0.4 fold. In another example, treatment reduces a subject's ferritin levels by about 0.5 fold. In these examples, treatment reduces a subject's ferritin levels by fold change relative to the subject's baseline ferritin level.

Methods to determine the level of the inflammatory biomarkers disclosed herein are known in the art. In an example, the level of a particular marker is determined in a sample obtained from a patient or subject (e.g. a blood sample, plasma sample, or serum sample). For example, the level of an inflammatory biomarker according to the present disclosure is determined in a plasma sample. In another example, the level of an inflammatory biomarker is determined in a serum sample.

In an example, the level of an inflammatory biomarker is determined by measuring the level of protein expression in a sample obtained from a subject. For example, inflammatory biomarker levels can be measured in a sample using antibody based immunoassays, such an Enzyme-Linked Immunosorbent (ELISA) assay, a multiplex immunoassays, for example, a Luminex assay (see, e.g. Cook et al. Methods. 158: 27-32. 2019), or a fluorescent bead-based immunoassay. In these examples, the level of inflammatory biomarker is expressed pg/mL. In another example, the level of inflammatory biomarker is expressed as fold change relative to an appropriate control. In another example, the level of an inflammatory biomarker is determined by measuring the level of gene expression in a sample obtained from a subject. For example, inflammatory biomarker levels can be measured in a sample using molecular based assays, such a qualitative polymerase chain reaction (PCR)-based assay, or a multiplex PCR assay, for example, a Luminex assay (see, e.g. Cook et al. Methods. 158: 27-32. 2019). In an example, the level of gene expression of an inflammatory biomarker is expressed as fold change relative to an appropriate control. For example, the fold change is calculated as log 2(fold-change).

In an examples, the level of multiple inflammatory biomarkers are measured in a single sample. In another example, the level of multiple inflammatory biomarkers are measured in separate samples. In an example, the level of biomarker is measured before treatment with MLPSCs. In another example, the level of biomarker is measured after treatment with MLPSCs. In another example, the level of biomarker is measured at baseline, and is monitored over time to determine whether a subject requires higher or more prolonged dosing.

The level of inflammatory biomarkers can be compared between samples to determine whether the level of inflammatory biomarker has reduced. In these examples, samples can be assessed to determine whether inflammation has reduced and, whether a reduction in inflammation is durable. In an example, a durable reduction in inflammation is determined based on an observed reduction in inflammation from baseline in at least two samples post administration of cell therapy.

### Mesenchymal Lineage Precursor Cells

As used herein, the term “mesenchymal lineage precursor or stem cell (MLPSC)” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. For the avoidance of doubt, a “mesenchymal lineage precursor cell” refers to a cell which can differentiate into a mesenchymal cell such as bone, cartilage, muscle and fat cells, and fibrous connective tissue.

The term “mesenchymal lineage precursor or stem cells” includes both parent cells and their undifferentiated progeny. The term also includes mesenchymal precursor cells, multipotent stromal cells, mesenchymal stem cells (MSCs), perivascular mesenchymal precursor cells, and their undifferentiated progeny.

Mesenchymal lineage precursor or stem cells can be autologous, allogeneic, xenogenic, syngenic or isogenic. Autologous cells are isolated from the same individual to which they will be reimplanted. Allogeneic cells are isolated from a donor of the same species. Xenogenic cells are isolated from a donor of another species. Syngenic or isogenic cells are isolated from genetically identical organisms, such as twins, clones, or highly inbred research animal models.

In an example, the mesenchymal lineage precursor or stem cells are allogeneic. In an example, the allogeneic mesenchymal lineage precursor or stem cells are culture expanded and cryopreserved.

Mesenchymal lineage precursor or stem cells reside primarily in the bone marrow, but have also shown to be present in diverse host tissues including, for example, cord blood and umbilical cord, adult peripheral blood, adipose tissue, trabecular bone and dental pulp. They are also found in skin, spleen, pancreas, brain, kidney, liver, heart, retina, brain, hair follicles, intestine, lung, lymph node, thymus, ligament, tendon, skeletal muscle, dermis, and periosteum; and are capable of differentiating into germ lines such as mesoderm and/or endoderm and/or ectoderm. Thus, mesenchymal lineage precursor or stem cells are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for mesenchymal lineage precursor or stem cells comprises at least about 0.1% or 0.5% or 1% or 2% or 5% or 10% or 15% or 20% or 25% or 30% or 50% or 75% mesenchymal lineage precursor or stem cells. In this regard, the term “population of cells enriched for mesenchymal lineage precursor or stem cells” will be taken to provide explicit support for the term “population of cells comprising X % mesenchymal lineage precursor or stem cells”, wherein X % is a percentage as recited herein. The mesenchymal lineage precursor or stem cells can, in some examples, form clonogenic colonies, e.g. CFU-F (fibroblasts) or a subset thereof (e.g., 50% or 60% or 70% or 70% or 90% or 95%) can have this activity.

In an example of the present disclosure, the mesenchymal lineage precursor or stem cells are mesenchymal stem cells (MSCs). The MSCs may be a homogeneous composition or may be a mixed cell population enriched in MSCs. Homogeneous MSC compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium. In an example, the MSCs are allogeneic. In an example, the MSCs are cryopreserved. In an example, the MSCs are culture expanded and cryopreserved.

In another example, the mesenchymal lineage precursor or stem cells are CD29+, CD54+, CD73+, CD90+, CD102+, CD105+, CD106+, CD166+, MHC1+ MSCs.

In an example, the mesenchymal lineage precursor or stem cells are culture expanded from a population of MSCs that express markers, including CD73, CD90, CD105 and CD166, and lack expression of hematopoietic cell surface antigens such as CD45 and CD31. For example, the mesenchymal lineage precursor or stem cells can be culture expanded from a population of MSCs that are CD73+, CD90+, CD105+, CD166+, CD45− and CD31−. In an example, the population of MSCs is further characterized by low levels of major histocompatibility complex (MHC) class I. In another example, the MSCs are negative for major histocompatibility complex class II molecules, and are negative for costimulatory molecules CD40, CD80, and CD86. In an example, the culture expansion comprises 5 passages.

In an example, the mesenchymal lineage precursor or stem cells are CD105+, CD156+, and CD45−. In another example, the mesenchymal lineage or precursor cells also express TNFR1 and suppress IL-2Ra expression on activated lymphocytes.

Isolated or enriched mesenchymal lineage precursor or stem cells can be expanded in vitro by culture. Isolated or enriched mesenchymal lineage precursor or stem cells can be cryopreserved, thawed and subsequently expanded in vitro by culture.

In one example, isolated or enriched mesenchymal lineage precursor or stem cells are seeded at 50,000 viable cells/cm2 in culture medium (serum free or serum-supplemented), for example, alpha minimum essential media (aMEM) supplemented with 5% fetal bovine serum (FBS) and glutamine, and allowed to adhere to the culture vessel overnight at 37° C., 20% O₂. The culture medium is subsequently replaced and/or altered as required and the cells cultured for a further 68 to 72 hours at 37° C., 5% O₂.

As will be appreciated by those of skill in the art, cultured mesenchymal lineage precursor or stem cells are phenotypically different to cells in vivo. For example, in one embodiment they express one or more of the following markers, CD44, NG2, DC146 and CD140b. Cultured mesenchymal lineage precursor or stem cells are also biologically different to cells in vivo, having a higher rate of proliferation compared to the largely non-cycling (quiescent) cells in vivo.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-1bright). Accordingly, an indication that cells are STRO-1+ does not mean that the cells are selected solely by STRO-1 expression. In one example, the cells are selected based on at least STRO-3 expression, e.g., they are STRO-3+(TNAP+).

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+(HSP-90B), CD45+, CD146+, 3G5+ or any combination thereof.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ cells are capable of differentiation into at least two different germ lines. Non-limiting examples of the lineages to which the STRO-1+ cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an example, mesenchymal lineage precursor or stem cells are obtained from a single donor, or multiple donors where the donor samples or mesenchymal lineage precursor or stem cells are subsequently pooled and then culture expanded.

Mesenchymal lineage precursor or stem cells encompassed by the present disclosure may also be cryopreserved prior to administration to a subject. In an example, mesenchymal lineage precursor or stem cells are culture expanded and cryopreserved prior to administration to a subject.

In an example, the present disclosure encompasses mesenchymal lineage precursor or stem cells as well as progeny thereof, soluble factors derived therefrom, and/or extracellular vesicles isolated therefrom. In another example, the present disclosure encompasses mesenchymal lineage precursor or stem cells as well as extracellular vesicles isolated therefrom. For example, it is possible to culture expand mesenchymal precursor lineage or stem cells of the disclosure for a period of time and under conditions suitable for secretion of extracellular vesicles into the cell culture medium. Secreted extracellular vesicles can subsequently be obtained from the culture medium for use in therapy.

The term “extracellular vesicles” as used herein, refers to lipid particles naturally released from cells and ranging in size from about 30 nm to as a large as 10 microns, although typically they are less than 200 nm in size. They can contain proteins, nucleic acids, lipids, metabolites, or organelles from the releasing cells (e.g., mesenchymal stem cells; STRO-1+ cells).

The term “exosomes” as used herein, refers to a type of extracellular vesicle generally ranging in size from about 30 nm to about 150 nm and originating in the endosomal compartment of mammalian cells from which they are trafficked to the cell membrane and released. They may contain nucleic acids (e.g., RNA; microRNAs), proteins, lipids, and metabolites and function in intercellular communication by being secreted from one cell and taken up by other cells to deliver their cargo.

### Culture Expansion of the Cells

In an example, mesenchymal lineage precursor or stem cells are culture expanded. “Culture expanded” mesenchymal lineage precursor or stem cells media are distinguished from freshly isolated cells in that they have been cultured in cell culture medium and passaged (i.e. sub-cultured). In an example, culture expanded mesenchymal lineage precursor or stem cells are culture expanded for about 4-10 passages. In an example, mesenchymal lineage precursor or stem cells are culture expanded for at least 5, at least 6, at least 7, at least 8, at least 9, at least 10 passages. For example, mesenchymal lineage precursor or stem cells can be culture expanded for at least 5 passages. In an example, mesenchymal lineage precursor or stem cells can be culture expanded for at least 5-10 passages. In an example, mesenchymal lineage precursor or stem cells can be culture expanded for at least 5-8 passages. In an example, mesenchymal lineage precursor or stem cells can be culture expanded for at least 5-7 passages. In an example, mesenchymal lineage precursor or stem cells can be culture expanded for more than 10 passages. In another example, mesenchymal lineage precursor or stem cells can be culture expanded for more than 7 passages. In these examples, stem cells may be culture expanded before being cryopreserved to provide an intermediate cryopreserved MLPSC population. In an example, compositions of the disclosure are prepared from an intermediate cryopreserved MLPSC population. For example, an intermediate cryopreserved MLPSC population can be further culture expanded prior to administration as is discussed further below. Accordingly, in an example, mesenchymal lineage precursor or stem cells are culture expanded and cryopreserved. In an embodiment of these examples, mesenchymal lineage precursor or stem cells can be obtained from a single donor, or multiple donors where the donor samples or mesenchymal lineage precursor or stem cells are subsequently pooled and then culture expanded. In an example, the culture expansion process comprises:
i. expanding by passage expansion the number of viable cells to provide a preparation of at least about 1 billion of the viable cells, wherein the passage expansion comprises establishing a primary culture of isolated mesenchymal lineage precursor or stem cells and then serially establishing a first non-primary (P1) culture of isolated mesenchymal lineage precursor or stem cells from the previous culture;
ii. expanding by passage expansion the P1 culture of isolated mesenchymal lineage precursor or stem cells to a second non-primary (P2) culture of mesenchymal lineage precursor or stem cells; and,
iii. preparing and cryopreserving an in-process intermediate mesenchymal lineage precursor or stem cells preparation obtained from the P2 culture of mesenchymal lineage precursor or stem cells; and,
iv. thawing the cryopreserved in-process intermediate mesenchymal lineage precursor or stem cells preparation and expanding by passage expansion the in-process intermediate mesenchymal lineage precursor or stem cells preparation.

In an example, the expanded mesenchymal lineage precursor or stem cell preparation has an antigen profile and an activity profile comprising:
i. less than about 0.75% CD45+ cells;
ii. at least about 95% CD105+ cells;
iii. at least about 95% CD166+ cells.

In an example, the expanded mesenchymal lineage precursor or stem cell preparation is capable of inhibiting IL2Rα expression by CD3/CD28-activated PBMCs by at least about 30% relative to a control.

In an example, culture expanded mesenchymal lineage precursor or stem cells are culture expanded for about 4-10 passages, wherein the mesenchymal lineage precursor or stem cells have been cryopreserved after at least 2 or 3 passages before being further culture expanded. In an example, mesenchymal lineage precursor or stem cells are culture expanded for at least 1, at least 2, at least 3, at least 4, at least 5 passages, cryopreserved and then further culture expanded for at least 1, at least 2, at least 3, at least 4, at least 5 passages before being administered or further cryopreserved.

In an example, the majority of mesenchymal lineage precursor or stem cells in compositions of the disclosure are of about the same generation number (i.e., they are within about 1 or about 2 or about 3 or about 4 cell doublings of each other). In an example, the average number of cell doublings in the present compositions is about 20 to about 25 doublings. In an example, the average number of cell doublings in the present compositions is about 9 to about 13 (e.g., about 11 or about 11.2) doublings arising from the primary culture, plus about 1, about 2, about 3, or about 4 doublings per passage (for example, about 2.5 doublings per passage). Exemplary average cell doublings in present compositions are any of about 13.5, about 16, about 18.5, about 21, about 23.5, about 26, about 28.5, about 31, about 33.5, and about 36 when produced by about 1, about 2, about 3, about 4, about 5, about 6, about 7, about 8, about 9, and about 10 passages, respectively.

The process of mesenchymal lineage precursor or stem cell isolation and ex vivo expansion can be performed using any equipment and cell handing methods known in the art. Various culture expansion embodiments of the present disclosure employ steps that require manipulation of cells, for example, steps of seeding, feeding, dissociating an adherent culture, or washing. Any step of manipulating cells has the potential to insult the cells. Although mesenchymal lineage precursor or stem cells can generally withstand a certain amount of insult during preparation, cells are preferably manipulated by handling procedures and/or equipment that adequately performs the given step(s) while minimizing insult to the cells.

In an example, mesenchymal lineage precursor or stem cells are washed in an apparatus that includes a cell source bag, a wash solution bag, a recirculation wash bag, a spinning membrane filter having inlet and outlet ports, a filtrate bag, a mixing zone, an end product bag for the washed cells, and appropriate tubing, for example, as described in U.S. Pat. No. 6,251,295, which is hereby incorporated by reference.

In an example, a mesenchymal lineage precursor or stem cell composition according to the present disclosure is 95% homogeneous with respect to being CD105 positive and CD166 positive and being CD45 negative. In an example, this homogeneity persists through ex vivo expansion; i.e. though multiple population doublings. In an example, the composition comprises at least one therapeutic dose of mesenchymal lineage precursor or stem cells and the mesenchymal lineage precursor or stem cells comprise less than about 1.25% CD45+ cells, at least about 95% CD105+ cells, and at least about 95% CD166+ cells. In an example, this homogeneity persists after cryogenic storage and thawing, where the cells also generally have a viability of about 70% or more.

In an example, compositions of the disclosure comprise mesenchymal lineage precursor or stem cells which express substantial levels of TNF-R1, for example greater than 13 pg of TNF-R1 per million mesenchymal lineage precursor or stem cells. In an example, this phenotype is stable throughout ex vivo expansion and cryogenic storage. In an example, expression of levels of TNF-R1 in the range of about 13 to about 179 pg (e.g. about 13 pg to about 44 pg) per million mesenchymal lineage precursor or stem cells is associated with a desirous therapeutic potential which also persists through ex vivo expansion and cryopreservation.

In an example, the culture expanded mesenchymal lineage precursor or stem cells express Tumor necrosis factor receptor 1 (TNF-R1) in an amount of at least 110 pg/ml. For example, the mesenchymal lineage precursor or stem cells can express TNF-R1 in an amount of at least 150 pg/ml, or at least 200 pg/ml, or at least 250 pg/ml, or at least 300 pg/ml, or at least 320 pg/ml, or at least 330 pg/ml, or at least 340 pg/ml, or at least 350 pg/ml.

In an example, the mesenchymal lineage precursor or stem cells express TNF-R1 in an amount of at least 13 pg/10⁶ cells. For example, the mesenchymal lineage precursor or stem cells express TNF-R1 in an amount of at least 15 pg/10⁶ cells, or at least 20 pg/10⁶ cells, or at least 25 pg/10⁶ cells, or at least 30 pg/10⁶ cells, or at least 35 pg/10⁶ cells, or at least 40 pg/10⁶ cells, or at least 45 pg/10⁶ cells, or at least 50 pg/10⁶ cells.

In another example, mesenchymal lineage precursor or stem cells disclosed herein inhibit IL-2Rα expression on T-cells. In an example, mesenchymal lineage precursor or stem cells can inhibit IL-2Rα expression by at least about 30%, alternatively at least about 35%, alternatively at least about 40%, alternatively at least about 45%, alternatively at least about 50%, alternatively at least about 55%, alternatively at least about 60.

In an example, compositions of the disclosure comprise at least one therapeutic dose of mesenchymal lineage precursor or stem cells which, for example, can comprise at least about 100 million cells or about 125 million cells.

### Modification of the Cells

In an example, mesenchymal lineage precursor or stem cells of the present disclosure may be altered in such a way that upon administration, lysis of the cell is inhibited. Alteration of an antigen can induce immunological non-responsiveness or tolerance, thereby preventing the induction of the effector phases of an immune response (e.g., cytotoxic T cell generation, antibody production etc.) which are ultimately responsible for rejection of foreign cells in a normal immune response. Antigens that can be altered to achieve this goal include, for example, MHC class I antigens, MHC class II antigens, LFA-3 and ICAM-1.

The mesenchymal lineage precursor or stem cells may also be genetically modified to express proteins of importance for the differentiation and/or maintenance of striated skeletal muscle cells. Exemplary proteins include growth factors (TGF-β, insulin-like growth factor 1 (IGF-1), FGF), myogenic factors (e.g. myoD, myogenin, myogenic factor 5 (Myf5), myogenic regulatory factor (MRF)), transcription factors (e.g. GATA-4), cytokines (e.g. cardiotropin-1), members of the neuregulin family (e.g. neuregulin 1, 2 and 3) and homeobox genes (e.g. Csx, tinman and NKx family).

Mesenchymal lineage precursor or stem cells of the disclosure can also be modified to carry or express an anti-viral agent or a thrombolytic agent. In an example, the agent is an anti-viral drug. In an example, the agent is anti-influenza. In an example, the agent is anti-SARS-COV (e.g. SARS-Cov2). An exemplary agent is remdesivir. In an example, the agent is a thrombolytic drug. Examples of thrombolytic agents include Eminase (anistreplase), Retavase (reteplase), Streptase (streptokinase, kabikinase). In an example, the thrombolytic agent is heparin.

Mesenchymal precursor or stem cells of the disclosure may be modified to carry an anti-viral or thrombolytic agent by culturing cells with the agent for a time and under conditions sufficient to allow the agent to be absorbed by the cells. In an example, the anti-viral or thrombolytic agent is added to the culture media of mesenchymal lineage precursor or stem cells disclosed herein. For example, mesenchymal lineage precursor or stem cells disclosed herein can be culture expanded in culture media comprising an anti-viral or thrombolytic agent.

In another example, the anti-viral or thrombolytic agent is a peptide. In an example, mesenchymal lineage precursor or stem cells are genetically modified to express a an anti-viral or thrombolytic peptide or a nucleic acid encoding the same. In an example, mesenchymal lineage precursor or stem cells are modified via contact with a viral vector in vitro. For example, virus can be added to cell culture medium. Non-viral methods of genetic modification may also be employed. Examples include plasmid transfer and the application of targeted gene integration through the use of integrase or transposase technologies, liposome or protein transduction domain mediated delivery and physical methods such as electroporation.

Efficiencies of genetic modification are rarely 100%, and it is usually desirable to enrich the population for cells that have been successfully modified. In an example, modified cells can be enriched by taking advantage of a functional feature of the new genotype. One exemplary method of enriching modified cells is positive selection using a selectable or screenable marker gene. “Marker gene” refers to a gene that imparts a distinct phenotype to cells expressing the marker gene and thus, allows such transformed cells to be distinguished from cells that do not have the marker. A selectable marker gene confers a trait for which one can “select” based on resistance to a selective agent (e.g., an antibiotic). A screenable marker gene (or reporter gene) confers a trait that one can identify through observation or testing, that is, by “screening” (e.g., B-glucuronidase, luciferase, GFP or other enzyme activity not present in untransformed cells). In an example, genetically modified mesenchymal lineage precursor or stem cells are selected based on resistance to a drug such as neomycin or colorimetric selection based on expression of lacZ.

### Compositions of the Disclosure

In one example of the present disclosure the mesenchymal lineage precursor or stem cells and/or progeny thereof and/or soluble factor derived therefrom are administered in the form of a composition. In one example, such a composition comprises a pharmaceutically acceptable carrier and/or excipient. Accordingly, in an example, compositions of the disclosure can comprise culture expanded mesenchymal lineage precursor or stem cells.

The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the present disclosure include those conventionally used, e.g., water, saline, aqueous dextrose, lactose, Ringer's solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. For example, such a media composition does not induce any adverse effects in a subject to whom it is administered.

Exemplary carriers and excipients do not adversely affect the viability of a cell and/or the ability of a cell to reduce, prevent or delay metabolic syndrome and/or obesity.

In one example, the carrier or excipient provides a buffering activity to maintain the cells and/or soluble factors at a suitable pH to thereby exert a biological activity, e.g., the carrier or excipient is phosphate buffered saline (PBS). PBS represents an attractive carrier or excipient because it interacts with cells and factors minimally and permits rapid release of the cells and factors, in such a case, the composition of the disclosure may be produced as a liquid for direct application to the blood stream or into a tissue or a region surrounding or adjacent to a tissue, e.g., by injection.

The mesenchymal lineage precursor or stem cells and/or progeny thereof and/or soluble factor derived therefrom can also be incorporated or embedded within scaffolds that are recipient-compatible and which degrade into products that are not harmful to the recipient. These scaffolds provide support and protection for cells that are to be transplanted into the recipient subjects. Natural and/or synthetic biodegradable scaffolds are examples of such scaffolds.

A variety of different scaffolds may be used successfully in the practice of the disclosure. Exemplary scaffolds include, but are not limited to biological, degradable scaffolds. Natural biodegradable scaffolds include collagen, fibronectin, and laminin scaffolds. Suitable synthetic material for a cell transplantation scaffold should be able to support extensive cell growth and cell function. Such scaffolds may also be resorbable. Suitable scaffolds include polyglycolic acid scaffolds, (e.g., as described by Vacanti, et al. J. Ped. Surg. 23:3-9 1988; Cima, et al. Biotechnol. Bioeng. 38:145 1991; Vacanti, et al. Plast. Reconstr. Surg. 88:753-9 1991); or synthetic polymers such as polyanhydrides, polyorthoesters, and polylactic acid.

In another example, the mesenchymal lineage precursor or stem cells and/or progeny thereof and/or soluble factor derived therefrom may be administered in a gel scaffold (such as Gelfoam from Upjohn Company).

The compositions described herein may be administered alone or as admixtures with other cells. The cells of different types may be admixed with a composition of the disclosure immediately or shortly prior to administration, or they may be co-cultured together for a period of time prior to administration.

In one example, the composition comprises an effective amount or a therapeutically or prophylactically effective amount of mesenchymal lineage precursor or stem cells and/or progeny thereof and/or soluble factor derived therefrom. For example, the composition comprises about 1×10⁵ stem cells to about 1×10⁹ stem cells or about 1.25×10³ stem cells to about 1.25×10⁷ stem cells/kg (80 kg subject). In an example, the composition comprises 2×10⁶ cells/kg. The exact amount of cells to be administered is dependent upon a variety of factors, including the age, weight, and sex of the subject, and the extent and severity of the disorder being treated.

In an example, 50×10⁶ to 200×10⁷ cells are administered. In other examples, 60×10⁶ to 200×10⁶ cells or 75×10⁶ to 150×10⁶ cells are administered. In an example, 75×10⁶ cells are administered. In another example, 150×10⁶ cells are administered.

In an example, the composition comprises greater than 5.00×10⁶ viable cells/mL. In another example, the composition comprises greater than 5.50×10⁶ viable cells/mL. In another example, the composition comprises greater than 6.00×10⁶ viable cells/mL. In another example, the composition comprises greater than 6.50×10⁶ viable cells/mL. In another example, the composition comprises greater than 6.68×10⁶ viable cells/mL.

In an example, the methods of the present disclosure encompass administering a total dose of 600 million cells. For example, a subject treated according to the present disclosure can receive multiple doses of an above referenced composition so long as the total dose of cells does not exceed 600 million cells. For example, the subject may receive 3 doses of 200 million cells. In an example, the total dose of cells is 500 million cells. In an example, the total dose of cells is 400 million cells. For example, the subject may receive 4 doses of 100 million cells. In an example, the subject receives 1 dose of 100 million cells at baseline followed by three doses of 100 million cells administered one per month over three months. In an example, a dose is 2×10⁶ cells/kg. In an example, a dose is 2×10⁶ cells/kg and the subject receives 2 doses or 3 doses. In an example, a dose is 2×10⁶ cells/kg and the subject receives more than 3 doses.

In an example, the mesenchymal lineage precursor or stem cells comprise at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% of the cell population of the composition.

Compositions of the disclosure may be cryopreserved. Cryopreservation of mesenchymal lineage precursor or stem cells can be carried out using slow-rate cooling methods or ‘fast’ freezing protocols known in the art. Preferably, the method of cryopreservation maintains similar phenotypes, cell surface markers and growth rates of cryopreserved cells in comparison with unfrozen cells.

The cryopreserved composition may comprise a cryopreservation solution. The pH of the cryopreservation solution is typically 6.5 to 8, preferably 7.4.

The cryopreservation solution may comprise a sterile, non-pyrogenic isotonic solution such as, for example, PlasmaLyte A™. 100 mL of PlasmaLyte A™ contains 526 mg of sodium chloride, USP (NaCl); 502 mg of sodium gluconate (C₆H₁₁NaO₇); 368 mg of sodium acetate trihydrate, USP (C₂H₃NaO₂·3H₂O); 37 mg of potassium chloride, USP (KCl); and 30 mg of magnesium chloride, USP (MgCl2·6H₂O). It contains no antimicrobial agents. The pH is adjusted with sodium hydroxide. The pH is 7.4 (6.5 to 8.0).

The cryopreservation solution may comprise Profreeze™. The cryopreservation solution may additionally or alternatively comprise culture medium, for example, aMEM.

To facilitate freezing, a cryoprotectant such as, for example, dimethylsulfoxide (DMSO), is usually added to the cryopreservation solution. Ideally, the cryoprotectant should be nontoxic for cells and patients, nonantigenic, chemically inert, provide high survival rate after thawing and allow transplantation without washing. However, the most commonly used cryoprotector, DMSO, shows some cytotoxicity. Hydroxylethyl starch (HES) may be used as a substitute or in combination with DMSO to reduce cytotoxicity of the cryopreservation solution.

The cryopreservation solution may comprise one or more of DMSO, hydroxyethyl starch, human serum components and other protein bulking agents. In one example, the cryopreserved solution comprises about 5% human serum albumin (HSA) and about 10% DMSO. The cryopreservation solution may further comprise one or more of methycellulose, polyvinyl pyrrolidone (PVP) and trehalose.

In one embodiment, cells are suspended in 42.5% Profreeze™/50% aMEM/7.5% DMSO and cooled in a controlled-rate freezer.

The cryopreserved composition may be thawed and administered directly to the subject or added to another solution, for example, comprising HA. Alternatively, the cryopreserved composition may be thawed and the mesenchymal lineage precursor or stem cells resuspended in an alternate carrier prior to administration.

In an example, cellular compositions of the disclosure can comprise Plasma-Lyte A, dimethyl sulfoxide (DMSO) and human serum albumin (HSA). For example, compositions of the disclosure may comprise Plasma-Lyte A (70%), DMSO (10%), HSA (25%) solution, the HSA solution comprising 5% HSA and 15% buffer.

In an example, the compositions described herein may be administered as a single dose.

In some examples, the compositions described herein may be administered over multiple doses. For example, at least 2, at least 3, at least 4 doses. In other examples, compositions described herein may be administered over at least 5, at least 6, at least 7, at least 8, at least 9, at least 10 doses.

In one example, the mesenchymal lineage precursor or stem cells can be culture expanded prior to administration to a subject. Various methods of cell culture are known in the art. In an example, mesenchymal lineage precursor or stem cells are culture expanded for about 4-10 passages. In an example, mesenchymal lineage precursor or stem cells are culture expanded for at least 4, at least 5, at least 6, at least 7, at least 8, at least 9, at least 10 passages. In an example, mesenchymal lineage precursor or stem cells are culture expanded for at least 5 passages. In these examples, stem cells may be culture expanded before being cryopreserved.

In an example, mesenchymal lineage precursor or stem cells are culture expanded in a serum free medium prior to administration.

In some examples, the cells are contained within a chamber that does not permit the cells to exit into a subject's circulation but permits factors secreted by the cells to enter the circulation. In this manner soluble factors may be administered to a subject by permitting the cells to secrete the factors into the subject's circulation. Such a chamber may equally be implanted at a site in a subject to increase local levels of the soluble factors.

In an example, mesenchymal lineage precursor or stem cells may be administered systemically. In an example, mesenchymal lineage precursor or stem cells may be administered to the subjects airway. In an example, mesenchymal lineage precursor or stem cells may be administered to the lung(s) of a subject. In another example, compositions of the disclosure are administered intravenously. In another example, compositions are administered intravenously and to the subjects airway.

In an example, mesenchymal lineage precursor or stem cells are administered once weekly. For example, mesenchymal lineage precursor or stem cells can be administered once weekly every two weeks. In another example, mesenchymal lineage precursor or stem cells are administered twice weekly. In an example, mesenchymal lineage precursor or stem cells can be administered once monthly. In an example, two doses of mesenchymal lineage precursor or stem cells are administered once weekly over two weeks. In another example, two doses of mesenchymal lineage precursor or stem cells are administered once weekly every two weeks. In another example, four doses of mesenchymal lineage precursor or stem cells are administered over two weeks before subsequent doses are administered monthly. In an example, two doses of mesenchymal lineage precursor or stem cells can be administered once weekly every two weeks before subsequent doses are administered once monthly. In an example, four doses are administered monthly.

In an example, compositions of the disclosure comprise a “clinically proven effective” amount of MLPSC. In an example, compositions of the disclosure comprise a “clinically proved effective” amount of MSCs.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

The following specific examples are to be construed as merely illustrative, and not limitative of the remainder of the disclosure in any way whatsoever. Without further elaboration, it is believed that one skilled in the art can, based on the description herein, utilize the present invention to its fullest extent.

### EXAMPLES

### Ex-Vivo Culture-Expanded Adult Allogeneic Bone Marrow Derived Mesenchymal Stem Cells (MSCs), for the Treatment of Acute Respiratory Distress Syndrome (ARDS)

### Composition

The composition is comprised of culture-expanded mesenchymal stem cells (ceMSC) isolated from the bone marrow of healthy adult donors. The final composition comprises ceMSC formulated in Plasma-Lyte A, dimethyl sulfoxide (DMSO) and human serum albumin (HSA).

### Objectives

To determine:
Safety
Improvement in survival.

### Subjects

Patients characterized as having moderate COVID-19 related ARDS received mesenchymal stem cells (intravenous; 2 million cells per kg) or control therapy. 125 patients were <65 years old (i.e. the intention to treat (ITT) population; Table 1). 58 of the ITT population received cell therapy and 67 received control therapy. Some ITT patients were identified as having mild ARDS and/or low levels of inflammation as characterized by circulating CRP <4. These patients were excluded from the per protocol (PP) patient population. Accordingly, the PP population was reduced to 89 (from 125) with 38 receiving cell therapy and 51 receiving control therapy. Furthermore, the PP population represents the study population with consistently the most severe disease as they all had moderate to severe ARDS.

TABLE 1
Baseline Summary Data: Intent to Treat Patients Pre-Specified Age <65 and ≥65.

| | ITT Patients <65 years | | | | ITT Patients ≥65 years | | | |
|---|---|---|---|---|---|---|---|---|
| | REM Mean | | Control Mean | | REM Mean | | Control Mean | |
| Category | n = 58 | | n = 67 | | n = 54 | | n = 43 | |
| Sex (%) | 76% | | 70% | | 65% | | 65% | |
| Male | 24% | | 30% | | 35% | | 35% | |
| Female | | | | | | | | |
| Age (Yrs) | 52 | (9.9) | 51 | (9.8) | 72 | (5.7) | 73 | (5.5) |
| BMI (kg/m²) | 34.1 | (7.7) | 36.6 | (8.2) | 32 | (7) | 32 | (6) |
| CRP (mg/L) | 29.8 | (58.8) | 19.5 | (17.5) | 17.2 | (27.8) | 26.4 | (51.9) |
| PF Ratio | 163 | (79) | 144 | (85) | 132 | (50) | 150 | (54) |
| ARDS Severity | 17.%, 48%, | | 9.%, 48%, | | 13.%, 57%, | | 14%, 67%, | |
| (mild, moderate, | 24% | | 37% | | 28% | | 14% | |
| severe) | (11% missing | | (6% missing | | (2% missing | | (5% missing | |
| | or no ARDS) | | or no ARDS) | | or no ARDS) | | or no ARDS) | |
| SOFA Score | 6.3 | (2.4) | 6.6 | (1.8) | 6.3 | (2) | 6.4 | (1.9) |

| | | | | |
|---|---|---|---|---|
| Any Steroids at | 67% | 84% | 98% | 93% |
| Baseline | | | | |
| Dexamethasone | 50% | 67% | 78% | 67% |
| at Baseline | | | | |
| Remdesivir at | 62% | 63% | 72% | 74% |
| Baseline | | | | |
| Anti-IL6 at | 3% | 4% | 7% | 5% |
| Baseline | | | | |

### Analysis

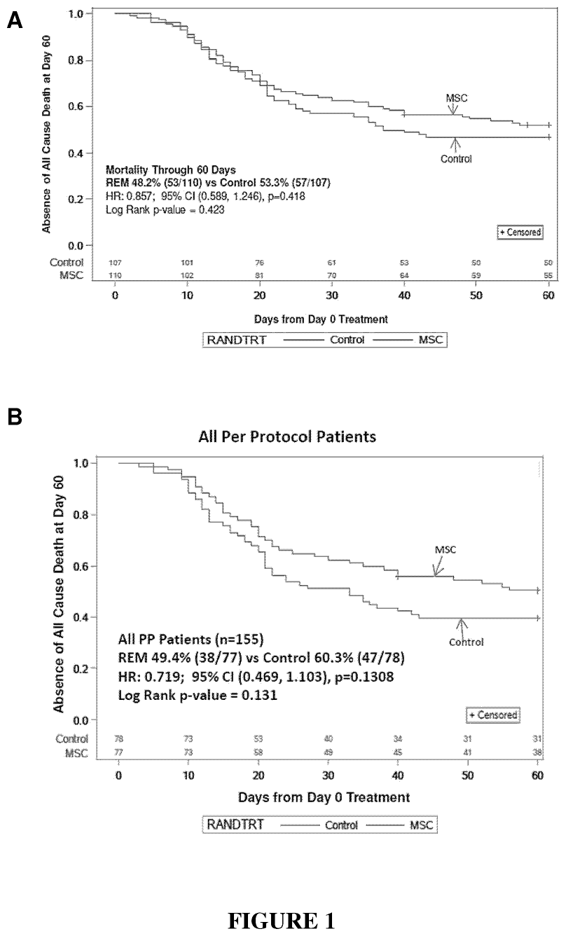

Cell therapy provides some degree of protection against death in all patients at day 60 (FIG. 1).

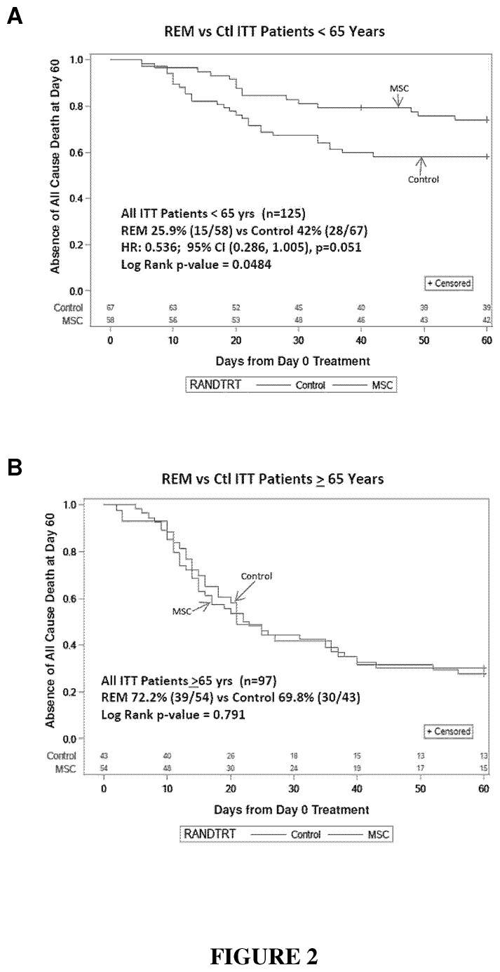

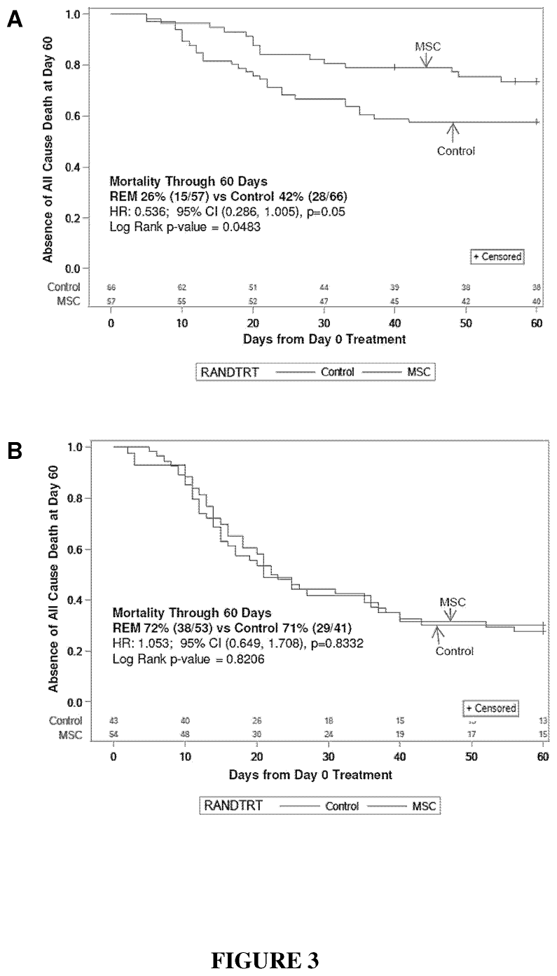

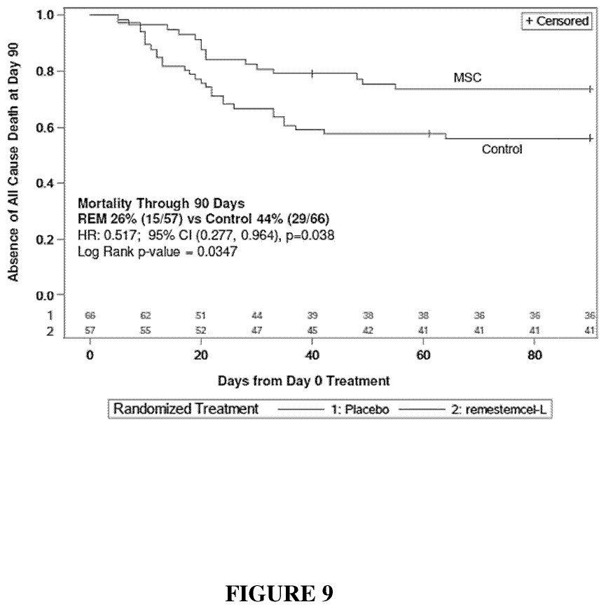

However, further analysis of treated patients surprisingly revealed that cell therapy provides significant protection against death at day 60 in patients less than 65 years of age (FIGS. 2, 3 and 9; A) relative to patients greater than 65 years of age (FIGS. 2, 3 and 9; B). Significantly, the protection against death at day 60 in patients less than 65 years of age was evident in both intention to treat (ITT) patients (38.3% reduction in death vs control; p=0.0484; FIG. 2) and per protocol (PP) patients (43.3% reduction in death vs control; p=0.0285; FIG. 3). As also shown in FIGS. 2 and 3, a greater reduction in death was observed in the PP patients compared with the ITT patients. This is an unexpected outcome as PP patients had consistently more severe disease than the ITT patients. These data suggest that subjects with more severe ARDS such as moderate to severe ARDS may respond better to treatment with cell therapy.

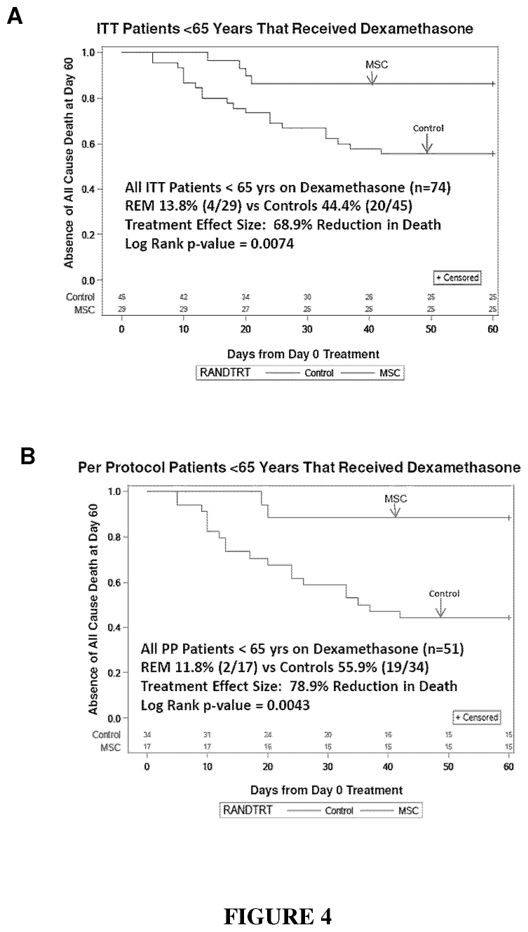

Significant protection against death was also observed in patients less than 60 years of age and receiving dexamethasone at baseline further supporting the rationale for selecting ARDS patients less than 65 years of age for treatment with cell therapy, in particular when the subjects are also receiving a corticosteroid (FIG. 4: A, ITT patients 68.9% reduction in death, p=0.0074; B, PP patients, 78.9% reduction in death, p=0.0043).

Patients less than 65 years are generally expected to have a higher health status than patients greater than 65 years of age. Despite their higher health status, these patients were dying from ARDS, an outcome that surprisingly improved following treatment relative to patients greater than 65 years of age. These data support the identification of an ARDS patient population that responds well to cell therapy, in particular in view of the protection provided against death in these patients.

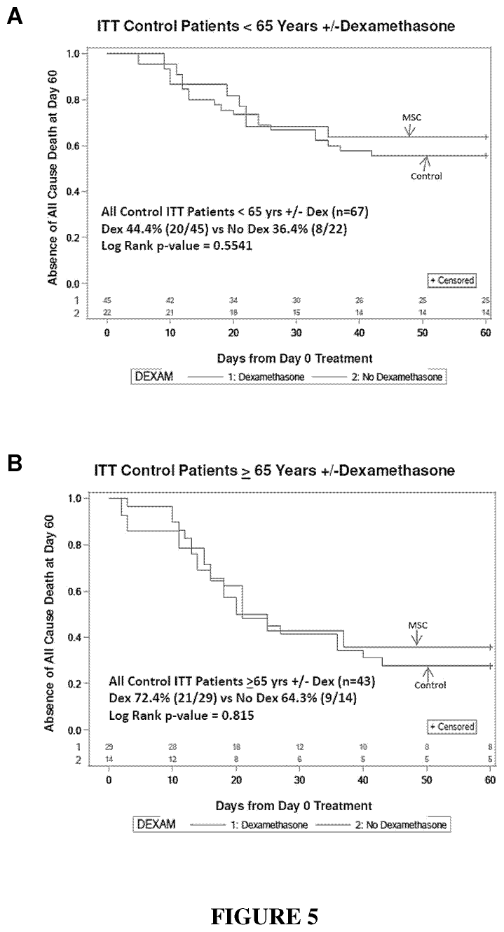

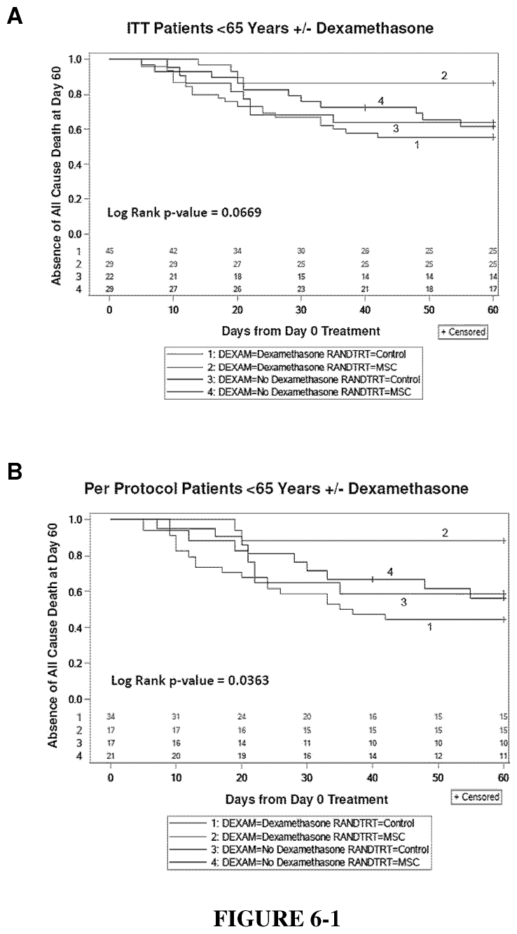

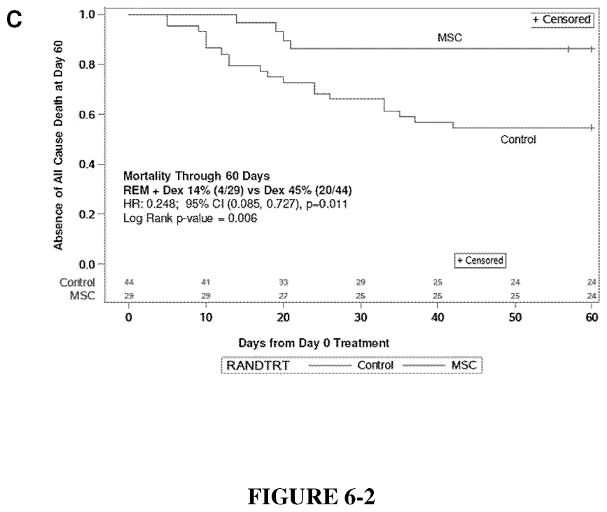

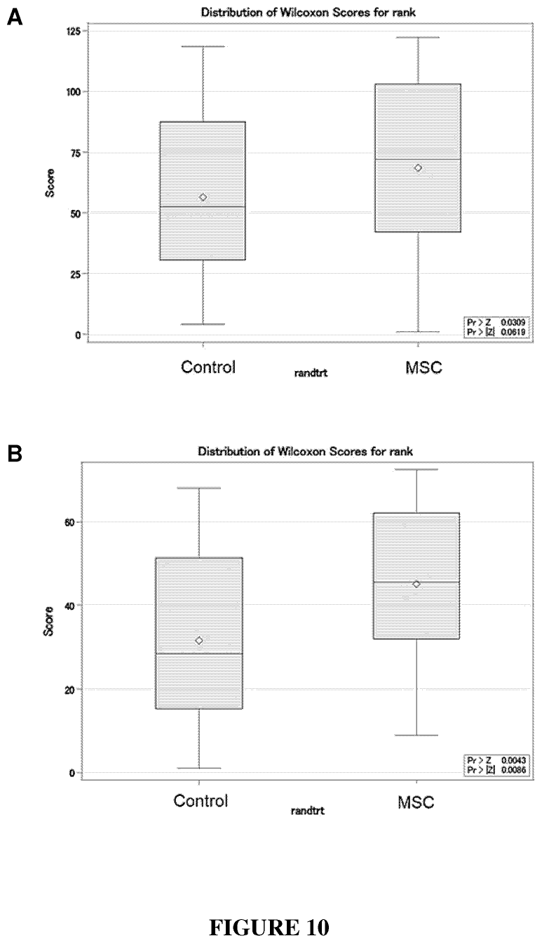

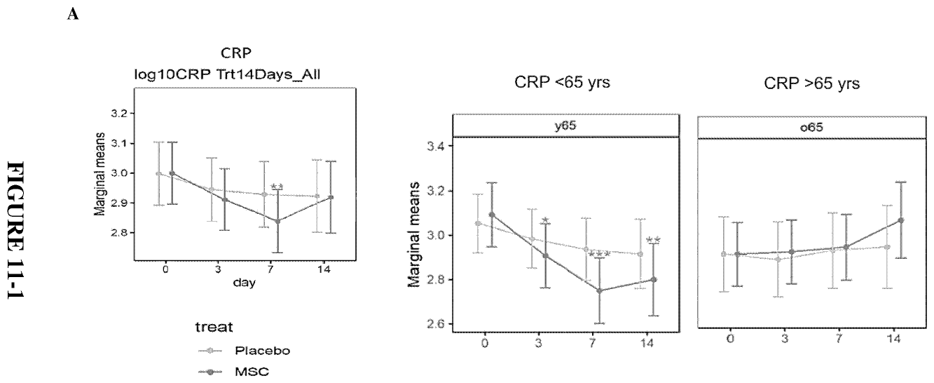

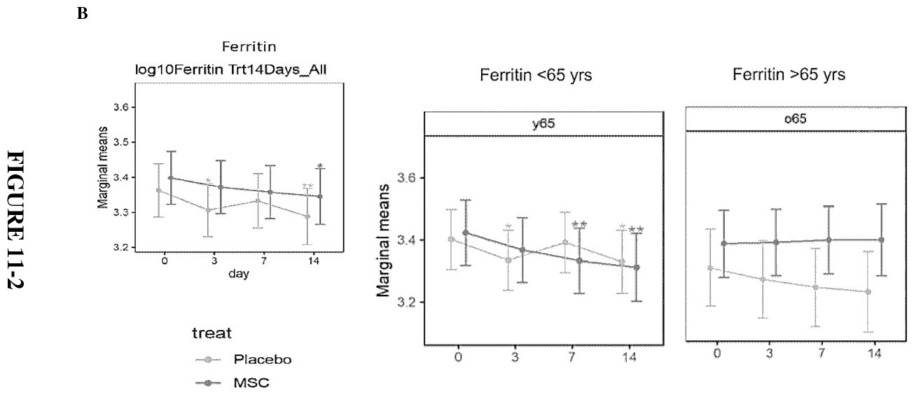

Analysis of the control population revealed that administration of dexamethasone at baseline does not reduce mortality in control patients (FIG. 5: A: All control ITT patients <65 years of age, p=0.554; B: All control ITT patients ≥65 years of age, p=0.815). Surprisingly, this lack of protection against death at day 60 was remedied in patients treated with cell therapy and dexamethasone (FIG. 4), with more than additive protection against death being observed in patients treated with cell therapy and dexamethasone (FIGS. 6, 7 and 8). Significantly, the observed synergy between cell therapy and dexamethasone was evident in both ITT patients (p=0.0669) and PP patients (p=0.0363). Combination of cell therapy with Dexamethasone was also synergistic in increasing days alive off ventilator through 60 Days in exploratory analysis of patients <65 (FIG. 10).

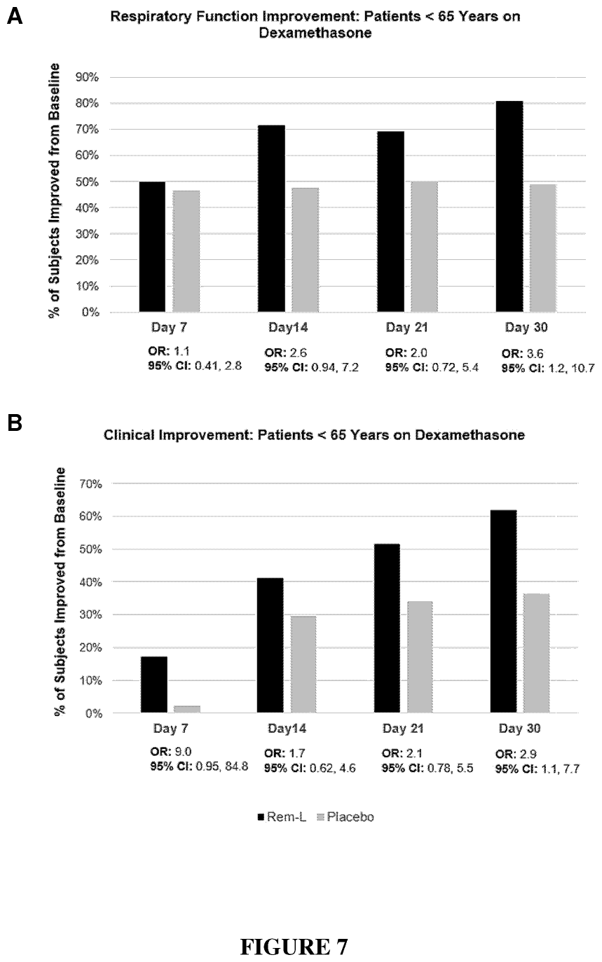

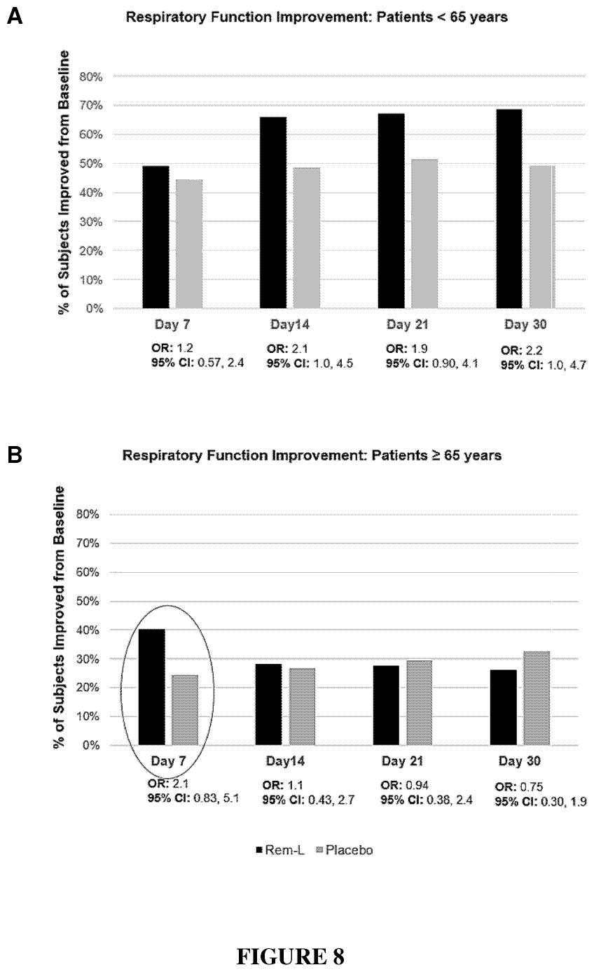

Protection against death for patients less than 65 years of age is summarized in FIG. 6. No protection was afforded to control patients or patients administered dexamethasone alone. Treatment with cell therapy imparted some protection against death and this was significantly increased by treatment with cell therapy and dexamethasone (see also FIG. 4). Improvements in both clinical and respiratory function were noted following the combination of cell therapy with Dexamethasone (FIGS. 7 and 8). This combination also increased the number of ventilator free days patients remained alive (FIG. 10).

These data provide a compelling rationale for selecting patients with ARDS that are less than 65 years of age for treatment with cell therapy and, in particular, treatment with cell therapy and a corticosteroid such as dexamethasone. These data also suggest significant improvement in treatment outcome can be achieved in ARDS patients by selecting ARDS patients less than 65 years old that are taking a corticosteroid and administering cell therapy to these patients.

### Biomarker Analysis

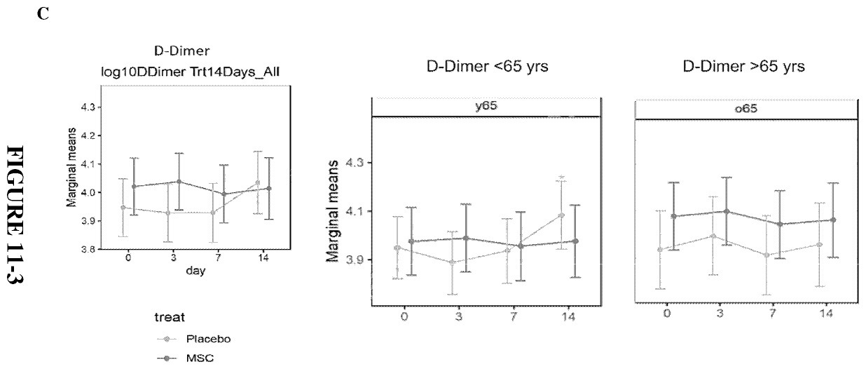

Levels of inflammatory biomarkers were measured in 107 treated patients and 106 controls (Treated <65 n=55; Control <65 n=65; Treated >65 n=52; Control >65 n=41) at baseline and after treatment with cell therapy (fixed dosing regimen of 2 million (2×10⁶) cells/kg×2 doses within 3-5 days). Cell therapy significantly reduced CRP levels at day 3, 7 and 14 (FIG. 11A) and ferritin levels at day 7 and day 14 (FIG. 11B) in patients <65. Cell therapy prevented a significant increase in D-dimer levels in all treated patients (FIG. 11C).

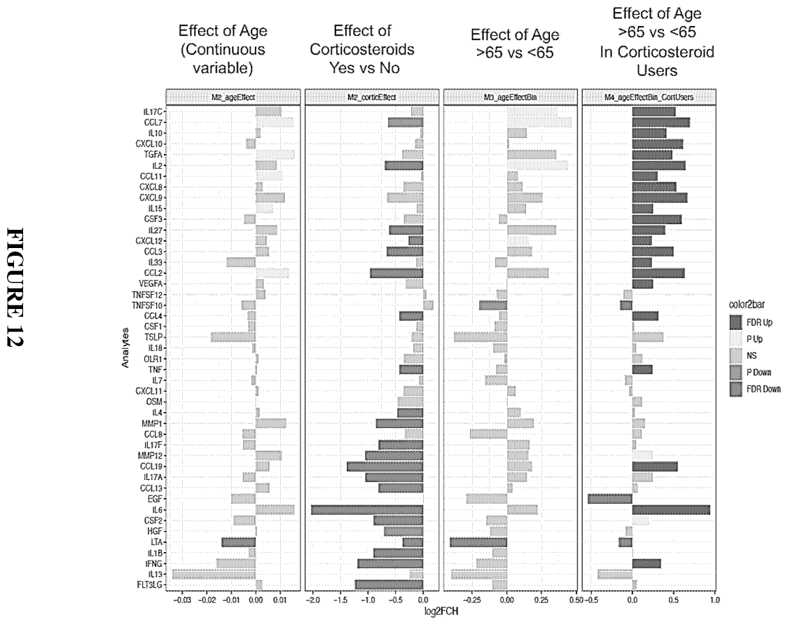

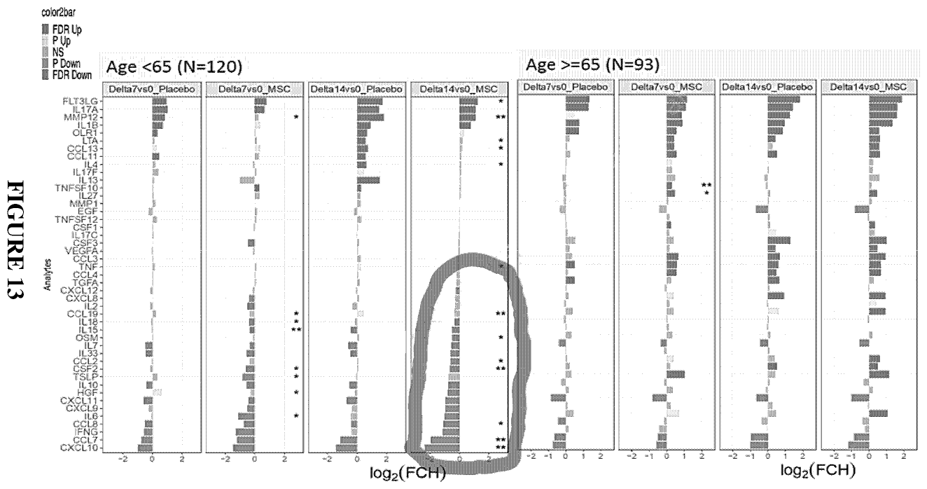

Comparing inflammatory biomarkers levels between treated subjects <65 years old and >65 years old provided a unique opportunity to identify measurable criteria for selecting patients for treatment. Surprisingly, as shown in FIGS. 12 and 13, age >65 years old appears to be associated with increased inflammatory activity at baseline, regardless of whether patients were on corticosteroids or not. Indeed, analysis of all patients on corticosteroids at baseline revealed >5-fold higher inflammatory activity (cytokines/chemokines) in patients >65 years old compared to those <65 years old.

FIG. 12 shows that patients older than 65 had higher baseline levels of inflammatory cytokines/chemokines than those <65, in particular:
(i) CCR2-binding chemokines (including CXCL10/IP10 and CXCL9) and CXCR3-binding chemokines (including CCL2, CCL3, and CCL7/MCP3). This group of chemokines are indicative of increased neutrophil and macrophage influx into lungs.
(ii) IL-6 and IL-8, which are indicative of increased macrophage inflammation and augmented neutrophil migration to lungs.
(ii) CCL19 and IL-2, which are indicative of T cell activation/proliferation and apoptotic death.

Other inflammatory biomarkers that were increased in >65 patients included TGF-alpha, TNF, CXCL8, G-CSF/CSF-3, and IL17C (FIGS. 12 and 13).

Patients <65 expressed the same inflammatory pathways as >65 patients, albeit at a lower baseline level. In <65 patients, cell therapy reduced inflammatory markers, in particular: associated with COVID-19 ARDS severity in those <65, notably:
CCR2−and CXCR3-binding chemokines, likely resulting in reduced neutrophil and macrophage influx into lungs;
IL-6/IL-8/TNF/IL-18, indicative of reduced IL-18-driven inflammasome, and reduced macrophage activation/neutrophil homing to lung;
reduced CCL19, and IL-4/IL-13/GM-CSF indicative of reduced T cell influx and activation.

In summary:
Inflammatory pathways were down regulated in treated patients <65 years old (FIGS. 12 and 13) and this corresponded with improved prognosis (FIGS. 2-4) and durable improvement in respiratory function (FIG. 8);
Improved respiratory function was observed in treated patients >65 years old at day 7 but was not sustained (FIG. 8); subsequent biomarker analysis revealed that these patients had a higher baseline level of inflammation (FIGS. 12 and 13);
Biomarker analysis revealed that similar inflammatory pathways are involved in ARDS patients regardless of age (<65 vs >65 years old).

### Safety

There were no infusion related adverse events.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

The present application claims priority from AU2021901214 filed 23 Apr. 2021, AU2021902180 filed 15 Jul. 2021, AU2022900260 filed 9 Feb. 2022 and AU2022900372 filed 18 Feb. 2022, the disclosures of which are incorporated herein by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

1. A method of treating or preventing Acute Respiratory Distress Syndrome (ARDS) in a human subject in need thereof, the method comprising administering to the subject a corticosteroid and a composition comprising mesenchymal lineage precursor or stem cells (MLPSCs).

2. The method of claim 1, wherein the subject is less than 65 years old.

3. A method of treating or preventing Acute Respiratory Distress Syndrome (ARDS) in a human subject in need thereof, the method comprising administering to the subject a composition comprising mesenchymal lineage precursor or stem cells (MLPSCs), wherein the subject is less than 65 years old and is taking a corticosteroid.

4. A method of treating or preventing Acute Respiratory Distress Syndrome (ARDS) in a human subject in need thereof, the method comprising selecting a subject with ARDS who is less than 65 years old and, administering to the subject a composition comprising mesenchymal lineage precursor or stem cells (MLPSCs).

5. The method according to any one of claims 1 to 4, wherein the ARDS is moderate or severe.

6. The method of claim 4, wherein the method comprises selecting a subject that is also taking a corticosteroid.

7. The method according to any one of claims 3 to 6, further comprising administering a corticosteroid.

8. The method according to any one of claims 1 to 7, wherein the subject is less than 60 years old.

9. The method according to any one of claims 1 to 3 or 5 to 7, wherein the subject is between 18 and 65 years old or between 18 and 60 years old.

10. The method according to any one of claims 1 to 3 or 5 to 9, wherein the corticosteroid is dexamethasone.

11. The method according to any one of claims 1 to 10, wherein the ARDS is caused by a viral infection such as a rhinovirus, an influenza virus, a respiratory syncytial virus (RSV) or a coronavirus.

12. The method of claim 11, wherein the viral infection is caused by a coronavirus.

13. The method of claim 12, wherein the coronavirus is Severe Acute Respiratory Syndrome coronavirus (SARS-CoV), Middle East Respiratory Syndrome coronavirus (MERS-CoV) or COVID-19.

14. The method according to any one of claims 1 to 13, wherein the ARDS is caused by a thrombosis such as a venous thrombosis or an arterial thrombosis.

15. The method according to any one of claims 1 to 14, wherein the ARDS is caused by a pulmonary embolism.

16. The method according to any one of claims 1 to 15, wherein a treated subjects risk of mortality is reduced after treatment.

17. The method according to any one of claims 1 to 15, wherein a treated subjects risk of mortality is reduced between 30 and 60%.

18. The method according to any one of claims 1 to 15, wherein a treated subject has improved 60 day survival.

19. The method of any one of claims 1 to 18, wherein the MLPSCs have been cryopreserved and thawed.

20. The method of any one of claims 1 to 19, wherein the MLPSCs are culture expanded from an intermediate cryopreserved MLPSCs population.

21. The method of claim 20, wherein the MLPSCs are culture expanded for at least about 5 passages.

22. The method of any one of claims 1 to 21, wherein the MLPSCs express at least 13 pg TNFR1 per million MLPSCs.

23. The method of any one of claims 1 to 22, wherein the MLPSCs express about 13 pg to about 44 pg TNFR1 per million MLPSCs.

24. The method of any one of claims 20 to 23, wherein said culture expansion comprises at least 20 or 30 population doublings.

25. The method according to any one of claims 1 to 24, wherein the MLPSCs are mesenchymal stem cells (MSCs).

26. The method according to any one of claims 1 to 25, wherein the MLPSCs are allogeneic.

27. The method according to any one of claims 1 to 26, wherein the MLPSCs are modified to carry or express an anti-viral drug or thrombolytic agent.

28. The method according to any one of claims 1 to 27 which comprises administering between 1×10⁷ and 2×10⁸ cells per dose.

29. The method according to any one of claims 1 to 28 which comprises administering about 1×10⁸ cells per dose.

30. The method according to claim 28 or claim 29, wherein the subject receives two doses.

31. The method according to claim 30, wherein the subject receives a second dose within 7 days of being administered a first dose.

32. The method of claim 31, wherein the second dose is administered 4 days after the first dose.

33. The method according to any one of claims 30 to 32, wherein a dose comprises 2×10⁶ cells/kg of body weight.

34. The method according to any one of claims 1 to 33, wherein the composition further comprises Plasma-Lyte A, dimethyl sulfoxide (DMSO), human serum albumin (HSA).

35. The method according to any one of claims 1 to 33, wherein the composition further comprises Plasma-Lyte A (70%), DMSO (10%), HSA (25%) solution, the HSA solution comprising 5% HSA and 15% buffer.

36. The method according to any one of claims 1 to 35, wherein the composition comprises greater than 6.68×10⁶ viable cells/mL.

37. The method according to any one of claims 1 to 36, wherein the subject is on a ventilator.

38. The method of claim 37, wherein the subject is taken off the ventilator after treatment.

39. The method of claim 38, wherein the subject is taken off a ventilator within 60 days of treatment.

40. The method according to any one of claims 1 to 39, wherein treatment decreases the level of at least one inflammatory biomarker(s) relative to baseline, wherein the at least one inflammatory biomarker(s) indicate:
(a) reduced neutrophil and macrophage influx into lungs;
(b) reduced inflammasome;
(c) reduced macrophage activation and neutrophil migration to lungs;
(d) reduced T cell influx and activation; or
(e) reduced circulating biomarkers of macrophage and neutrophil inflammation.

41. The method according to claim 40, wherein the inflammatory biomarker(s) is one or more of the following:
a CXCR3-binding chemokine, preferably CXCL10, and/or CXCL9;
CCR2-binding chemokine, preferably CCL2, CCL3, and/or CCL7;
IL-6;
IL-8;
TNF;
IL-18;
CCL19;
IL-4;
IL-13;
GM-CSF;
CRP; or
Ferritin.

42. The method according to any one of claims 1 to 41, wherein treatment reduces CRP and/or ferritin levels within 3 to 14 days of administering MLPSCs.

43. The method of claim 42, wherein respiratory function as defined by Berlin criteria is improved at day 14 and/or day 21.
